(19) United States
(12) Patent Application Publication
(10) Pub. No.: US 2016/0166191 A1
(43) Pub. Date: Jun. 16, 2016

(54) PROCESS, SYSTEM AND COMPUTER PROGRAM PRODUCT FOR EVALUATING PSYCHOLOGICAL STATUS

(71) Applicant: King Fahd University of Petroleum and Minerals, Dhahran (SA)

(72) Inventors: Ahmed Zafer AL-GARNI, Dhahran (SA); Wael Gamal Eldin ABDELRAHMAN, Dhahran (SA)

(73) Assignee: King Fahd University of Petroleum and Minerals, Dhahran (SA)

(21) Appl. No.: 14/568,150

(22) Filed: Dec. 12, 2014

(51) Int. Cl. A61B 5/16; G09B 5/00; A61B 5/00

(52) U.S. Cl. CPC A61B 5/165; A61B 5/7246; A61B 5/7282; A61B 5/0022; A61B 5/742; A61B 5/7475; G09B 5/00

(57) ABSTRACT

A process, a computer system and a computer program product for evaluating psychological status. In the process, a subject human is presented with a plurality of antonym pairs and a prescribed group of colors. Each color has been pre-assigned with a numerical code. The subject human is asked to arrange the colors in the prescribed group to reflect each word in the plurality of antonym pairs, therefore producing a plurality of subject arrangements of colors that each represents the subject human's perception of a word in an antonym pair. The result of the psychological status evaluation process is expressed as a standard deviation and/or a linear correlation coefficient, calculated based on comparisons of the plurality of subject arrangements of colors to a plurality of reference arrangements of colors. A computer system and a computer program product implementing the psychological status evaluation process are also provided.

### BACKGROUND OF THE INVENTION

1. Technical Field

The present invention relates to computer-implemented processes, systems and computer program products for evaluating the psychological status, emotional state and knowledge state of a person. More particularly, these computer-implemented processes, systems and computer program products analyze the person's arrangement of colors to reflect pairs of words with meanings that are opposite to each other.

2. Description of the Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

The effects of colors on mood, cognition and behavior have been well-documented. For example, there is strong statistical evidence that the color green triggers positive feelings. Another study shows that red and orange dominate in human imagination as stimulating (80%) while blue is pacifying to 98% of the tested population (Hettlarachchi A A, De Silva N. Colour associated emotional and behavioural responses: A study on the associations emerged via imagination. Built-Environment-Sri Lanka 2012 11(1): 21-27—incorporated herein by reference in its entirety). It has also been shown that emotional responses toward colors can be learned or nurtured. For example, the calming effect of the color white on certain people could stem from their religious or social backgrounds.

Psychologists and neuroscientists have often exploited the aforementioned effects of colors as a means to evaluate the psychological status of their patients and subjects. Color-based psychology/personality tests are typically in the form of questionnaires, and they usually associate a subject's color preferences and responses to colors with personality traits and emotional states. However, these tests can be subjective especially when color preference is made the primary metric for evaluation or when a test taker is asked to choose colors and/or adjectives to describe his or her personality.

Furthermore, a common criticism against personality tests that are in the market is that the respondents are often able to distort their responses. Emotive tests, in particular, could become prey to unreliable results to people striving to pick the answer they feel the best fitting of an ideal character and therefore not their true response. This is especially problematic in employment contexts and other contexts where important decisions are being made and there is an incentive to present oneself in a favorable manner.

Accordingly, there is a need for objective and accurate methods, processes and systems for evaluating a person's psychological status based on color schemes.

### BRIEF SUMMARY OF THE INVENTION

According to a first aspect, the present invention provides a computer-implemented process of evaluating the psychological status of a subject human. The computer-implemented process comprises comparing the subject human's arrangements of a prescribed group of colors to one or more reference arrangements of the prescribed group of colors by one or more human references. The subject human's arrangements of the prescribed group of colors represents the subject human's perception of at least one pair of words with opposite meanings. The subject human's arrangements of the prescribed group of colors include first and second subject arrangements of colors. The reference arrangements of the prescribed group of colors include first and second reference arrangements of colors. The comparing in the computer-implemented comprises the following steps instructing the subject human to arrange the prescribed group of colors to reflect a positive emotional state or a favorable word to obtain the first subject arrangement of colors, instructing the subject human to arrange the prescribed group of colors to reflect a negative emotional state that is opposite to the positive emotional state or a unfavorable word that is opposite to the favorable word to obtain the second subject arrangement of colors, associating each color in the prescribed group of colors with a numerical code to obtain first and second subject arrangements of numerical codes from the first and the second subject arrangements of colors and first and second reference arrangements of numerical codes from the first and the second reference arrangements of colors and deriving a standard deviation and/or a linear correlation coefficient based on the differences between the first and the second subject arrangements of numerical codes and the first and the second reference arrangements of numerical codes, respectively.

In one or more embodiments, the one or more human references in the computer-implemented process comprise a population of psychologically healthy humans.

In one or more embodiments, the first and the second subject arrangements of numerical codes and the first and the second reference arrangements of numerical codes are each arranged in an array.

In one or more embodiments, the differences are based on the differences in the locations of the numerical codes in the arrays.

In one or more embodiments of the computer-implemented process, a standard deviation of greater than 2 is indicative of an abnormal psychological status.

In one or more embodiments of the computer-implemented process, a linear correlation coefficient of less than 0.95 is indicative of an abnormal psychological status.

According to a second aspect, the present invention provides a non-transitory computer-readable storage medium having computer-readable instructions thereon which when executed by a computer case the computer to perform the computer-implemented process according to the first aspect of the invention.

According to a third aspect, the present invention provides a computer system having at least one processor module and at least one memory unit communicatively coupled to the processor module, the memory module configured to store one or more modules that, when executed by the processor module, are operative to perform the computer-implemented process according to the first aspect of the invention.

In one or more embodiments, the computer system further comprises one or more peripheral devices.

The foregoing paragraphs have been provided by way of general introduction, and are not intended to limit the scope of the following claims. The described embodiments, together with further advantages, will be best understood by reference to the following detailed description taken in conjunction with the accompanying drawings.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 is a flowchart of the process of evaluating a person's psychological status according to one or more embodiments

FIG. 2 illustrates a color data array of a subject human according to one or more embodiments

FIG. 3 illustrates a numerical code data array based upon the color data array of FIG. 2.

FIG. 4 illustrates a reference numerical code data array according to one or more embodiments FIG. 5 illustrates the calculation or computation of deviation values according to one or more embodiments.

FIG. 6 is a block diagram of the hardware components of a computer system upon which exemplary embodiments of the present invention may be implemented.

### DETAILED DESCRIPTION OF THE EMBODIMENTS

Referring now to the drawings, wherein like reference numerals designate identical or corresponding parts throughout the several views.

The present invention provides a method or process of evaluating a person's psychological status. In addition to psychological status, the method or process also provides an insight of the personality traits, emotional state and the knowledge state of the person.

Preferably, the process is computer-implemented but embodiments of the present invention are not so limited. Further, the process can be supported by computer and telecommunications networks such as LAN, WLAN, WAN, Internet and/or any other forms of wired or wireless, server-based or non-server-based networks. The process can be used by different groups of individuals and organizations, and in a wide range of context. For example, mental health agencies or mental health professionals such as licensed and trained psychologists, psychiatrists, counsellors and therapists can use the evaluation process to assess their clients and to assess the effectiveness of their other treatment methods (e.g. medication, psychotherapy, etc.). Career guidance centers and career counselors can apply the process to assess the suitability of a career path for a candidate. Individuals can use the process as a form of self-help or self-knowledge. Organizations such as airlines, militaries and police can include the evaluation process during their hiring process to assess prospective employees. The results and outcomes of the psychological status evaluation process disclosed herein can correlate with, for example, academic performance of students and work performance of employees.

For purposes of the present invention, the term “psychological status” is used synonymously with “psychological state”, “psychological condition”, “mental state” and “mental condition”, to refer to a person's state of mind, which can be current or chronic, under any of the domains including appearance, attitude and outlook, behavior, mood and affect, speech, thought process, thought content, perception, cognition, insight and judgment. Results of a test evaluating a person's psychological status can be an indication of a person's mental health and psychological disorder.

For purposes of the present invention, the term “emotional state” encompasses a person's mood at time of evaluation and the person's chronic emotional state, such as anxious, happy, disappointed and hopeful. A person's emotional state is subjective and conscious, and can be influenced by current events, personality and psychological status.

As used herein, “knowledge state” refers to the knowledge level, intelligence and intellectual capacity of a person.

Terms such as “first” and “second” are used to describe to various elements and features, such as set of words, order of colors, order of numerical codes, only for purposes of clarity and discriminating one element or feature from another element or feature. Therefore, the elements and features are not limited to these terms.

For purposes of the present invention, “order”, “arrangement” and “sequence” are used interchangeably to refer to a two-dimensional arrangement of data comprising one or more group of elements (e.g. colors or numerical codes) wherein one element comes after the other, horizontally or vertically. The data can be arranged systematically in the form of an array, usually in rows and columns, and usually from top to bottom for columns and from left to right for rows, but are not so limited.

As used herein, an “array” refers to a matrix or a two-dimensional data structure consisting of a collection of elements, each identifiable by at least one array index or key, which may be organized and processed with one or more algorithms of a computer, and stored in the memory of computer.

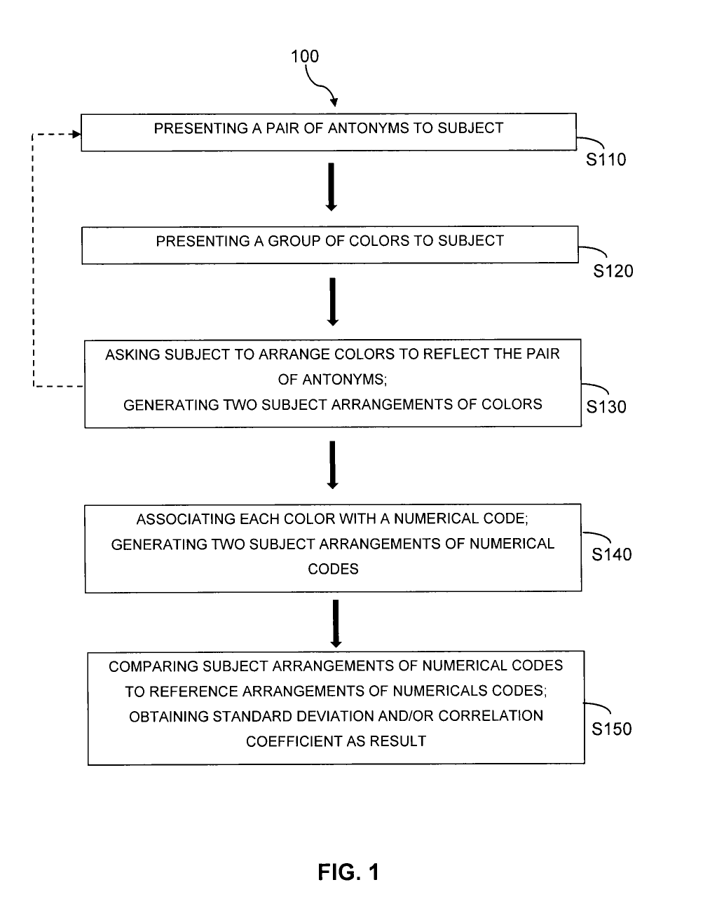

The psychological status evaluation process 100, as shown in the flowchart of FIG. 1, begins at step S110 wherein a subject human is presented with a pair of antonyms, ie. two words whose meanings are opposite. The antonym pair may consist of a first word that is generally favorable and a second word that is generally unfavorable and has an opposite meaning to the first word, for example, safety-danger, life-death, peace-war, wealth-poverty, health-illness, etc. Alternatively or additionally, the antonym pair may consist of a first word that describes a generally positive emotion and a second word that describes a generally negative emotion and has an opposite meaning to the first word, for example, happy-sad, calm-disturbed, joy-sorrow, love-hate, etc.

At step S120, the subject human is presented with a group of colors, such as red, blue, green, yellow, white, black, purple, orange, etc. These colors are selected to represent one part or one word of each antonym pair, based on available surveys of a “reference group of people” which are deemed psychologically and emotionally stable. Selecting which color goes with which antonym part can be based on available studies in the literature on the subject. For example, for the antonym pair “peace-war”, the following colors may be selected, in order, to represent “peace”: green, white, yellow, orange, red, and black.

In at least one embodiment, the colors in the prescribed group are selected and set based on a CIE (Commission Internationale de l'Eclairage) model of color spaces, such as CIE 1931 XYZ, CIELUV, CIELAB and CIEUVW, where colors are mathematically expressed as tuples of numbers, i.e. RGB tristimulus values. The CIE color model was developed to be completely independent of any device or other means of emission or reproduction and is based as closely as possible on how humans perceive color. The colors in the prescribed group should be different as reflected by the RGB tristimulus values or relative color spaces according to the CIE color model. Preferably the colors of a prescribed group of colors range across the spectrum of visible colors such that the CIE values of the colors are recognized as distinct colors by an average observer.

In an alternative embodiment, the CIE color model can be combined with other color models, such as CMYK, especially when the psychological status evaluation process is conducted in a printed format.

Preferably, the prescribed group of colors contains at least 3 colors for statistical purposes but no more than 10 colors, since the psychological status evaluation process 100 is designed to be a quick psychological test that is no more than 15 min long (from step S110 to step S150).

The antonym pair and the group of colors at steps S110 and S120 can be presented visually to the subject human on an output display device such as a computer monitor or a television (connected to a computing device), a display screen of a computer such as a personal computer, a desktop computer, a laptop, a tablet or a smartphone. Alternatively, the antonym pair and the group of colors at steps S110 and S120 are presented in printed format to the subject human, for example, on paper.

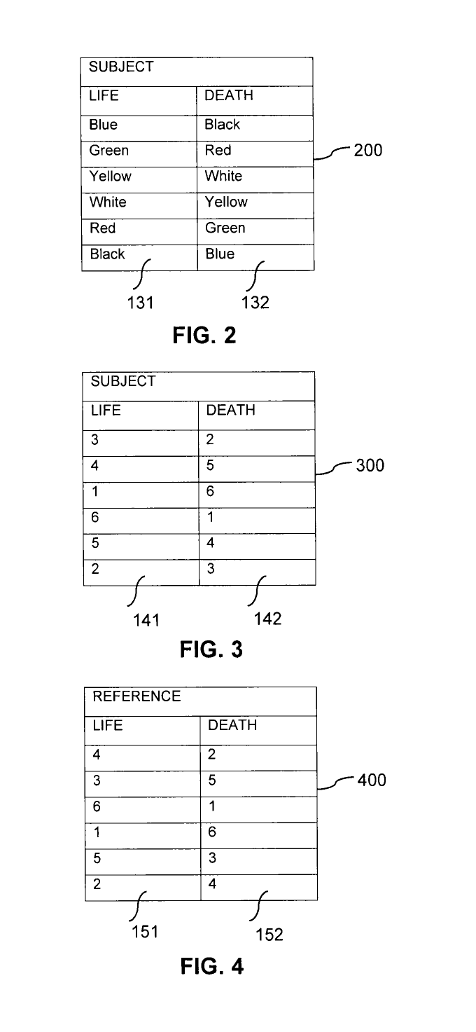

At step S130, the subject human is instructed to arrange the colors in the prescribed group to reflect the antonym pair. The instruction can be sent visually and/or orally, for example, via display screen and/or loudspeaker of a computer. The subject human can arrange the colors to reflect the two words in the antonym pair by, for example, typing responses using a keyboard, clicking and dragging color icons on the display screen of the computer with a mouse or by simply touching a display screen incorporated with touch technology with his or her finger. Accordingly, at step 130, a subject color data array 200 comprising two arrangements of colors is generated, for example, by a computer processor: the first arrangement of colors 131 represents the subject human's perception of the first word in the antonym pair while the second arrangement of colors 132 represents his or her perception of the second word in the antonym pair that has a meaning opposite to the meaning of the first word (see FIG. 2).

After step S130, the evaluation process 100 can return to repeat steps S110, S120 and S130 multiple times with multiple different pairs of antonyms. In one or more embodiments, for a quick evaluation lasting no longer than 15 min, 5 pairs of antonyms may be tested on a subject human. In alternative embodiments, for a more thorough and stringent evaluation, up to 20 pairs of antonyms may be tested, for example, 10, 12 and 15.

After all antonym pairs have been tested, the psychological status evaluation process 100 can proceed to steps S140 and S150, both of which require no further direct participation from the subject human and can be executed by a computer processor. At step 140, with each color in the prescribed group being pre-assigned with a numerical code, a subject numerical code data array 300 is generated, for example, by the computer processor. The subject numerical code data array 300 can comprise two arrangements of numerical codes corresponding to the first and second subject arrangements of colors 131, 132, respectively. The first subject arrangement of numerical codes 141 represents the subject human's perception of the first word in the antonym pair whereas the second subject arrangement of numerical codes 142 represents his or her perception of the second word in the antonym pair that has a meaning opposite to the meaning of the first word (see FIG. 3). Ideally, since arrangements of colors are used to reflect a pair of words with meanings that are directly opposite to each other, the first and second subject arrangements of numerical codes 141, 142 should be reverse of each other.

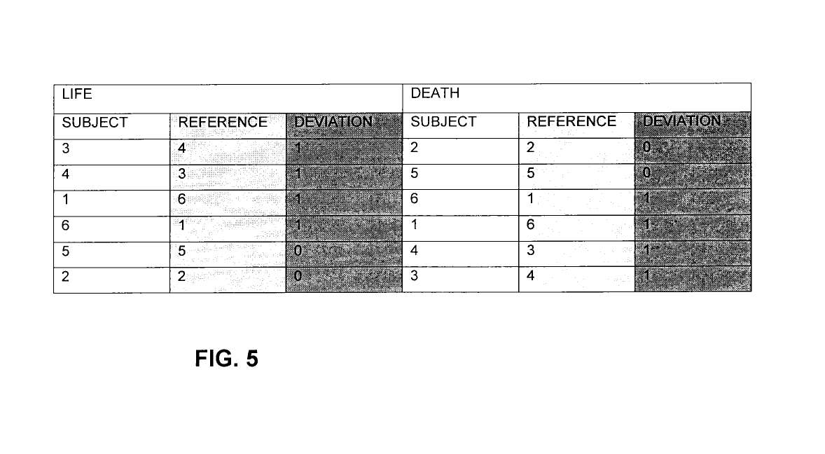

Next, at step S150 of the psychological status evaluation process 100, the subject numerical code data array 300 is compared to a reference numerical code data array 400. Shown in FIG. 4, the reference numerical code data array 400 consists of first and second reference arrangements of numerical codes 151, 152 derived statistically from a population of psychologically healthy individuals. The first and second reference arrangements of numerical codes 151, 152 are reverse of each other. To make the comparison, the deviation of each color in the prescribed group, each of which is represented by a numerical code, is quantified based on the distance between the positions or coordinates (x, y) of the color on the subject and reference numerical code data arrays 300 and 400, respectively, and for the same word in the antonym pair. For example, the color blue or its numerical code 3 has a coordinate of (1, 1) for the word “life” on the subject numerical code data array 300. The color blue has a coordinate of (1, 2) in the reference numerical code data array 400. The deviation of the color blue for the word “life” is (1, 2)−(1, 1)=1. Similarly, the deviation of the color black for the “death” is (1, 1)−(1, 1)=0.

The deviation values for all colors, based upon the examples given in FIGS. 3 and 4, are shown in FIG. 5. The result of the psychological status evaluation process 100 may be expressed, at step S150 of FIG. 1, as a standard deviation, s, calculated using the following Equation 1:

$$s = \sqrt{\frac{1}{n - 1} {\sum }_{i = 1}^{n} {( {x}_{i} - \overline{x} )}^{2}} \qquad ( Equation 1 )$$

where n is the number of colors or numerical codes; x₁, x₂, . . . and xₙ are the deviation values and x̅ is the deviation mean, which is 0.

In certain embodiments, the result of the psychological status evaluation process 100 may be expressed, at step S150 of FIG. 1, as a discrete linear correlation coefficient, r, computed using the following Equation 2:

$$r = \frac{n \sum xy - ( \sum x ) ( \sum y )}{\sqrt{n ( \sum {x}^{2} ) - {( \sum x )}^{2}} \sqrt{n ( \sum {y}^{2} ) - {( \sum y )}^{2}}} \qquad ( Equation 2 )$$

In one or more embodiments, the result of the psychological status evaluation process 100 may be expressed as both a standard deviation (s) and a linear correlation coefficient (r).

Accordingly, a standard deviation that is higher than a pre-set limit of standard deviation (e.g. >2) and/or a linear correlation coefficient that is lower than a pre-set limit of linear correlation coefficient (e.g. <0.95) may be indicative of abnormal psychological status, which is defined as unusual patterns of behavior, emotion and thought. A standard deviation that is higher than a pre-set limit of standard deviation and/or a linear correlation coefficient that is lower than a pre-set limit of linear correlation coefficient can reveal a certain degree of internal avoidance of one or more colors that may correlate with personality traits, emotional state at time of evaluation, past experiences and knowledge state of a subject human.

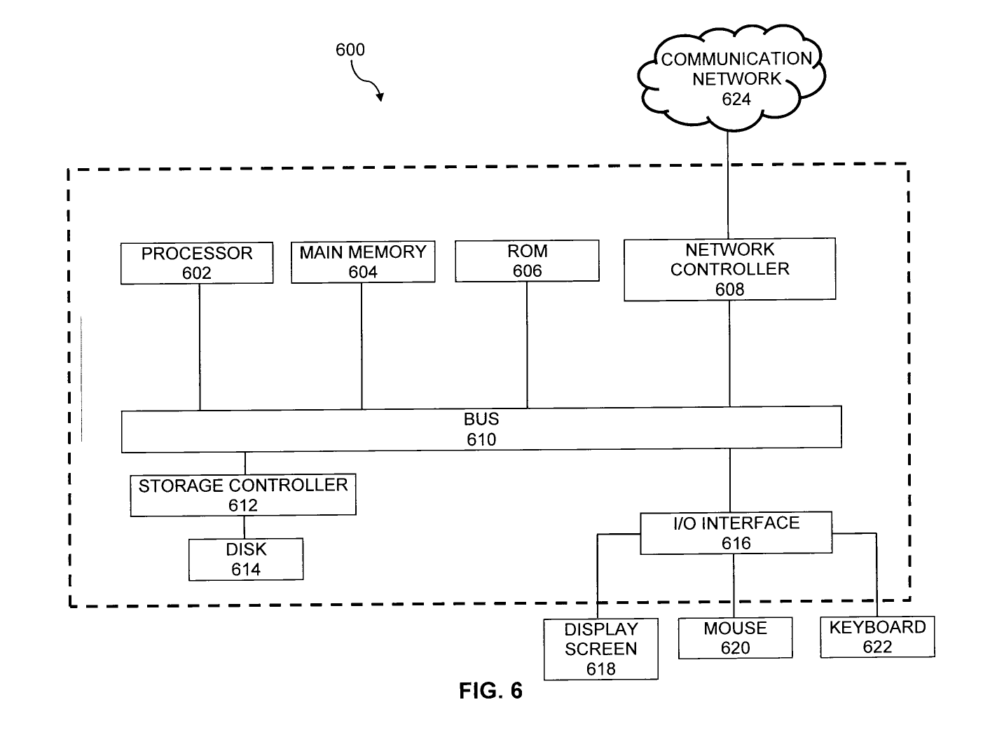

FIG. 6 illustrates a block diagram of the hardware components of a computer system 600 upon which one or more embodiments of the present invention may be implemented. The computer system 600 includes a bus 610 or other communication mechanism for communicating information, and a processor 602 coupled with the bus 610. The processor 602 is the primary component of the computer system 600 that carries out the instructions of a computer program or an algorithm, such as by presenting, receiving and processing information, performing arithmetical, statistical, logical operations of steps S110 through S160 of the psychology status evaluation process 100.

The computer system 600 also includes a main memory 604, such as a random access memory (RAM) or other dynamic storage device (e.g., dynamic RAM (DRAM), static RAM (SRAM), and synchronous DRAM (SDRAM)), coupled to the bus 610 for storing information and instructions to be executed by processor 602. In addition, the main memory 604 may be used for storing temporary variables or other intermediate information during the execution of instructions by the processor 602.

On the other hand, processes and instructions may also be stored on a storage medium disk 614 such as a hard drive (HDD) or portable storage medium or may be stored remotely. A database of standard deviations and/or linear correlation coefficients that are correlated with different psychological statuses, may also be stored on the storage medium disk 614. Further, the claimed advancements are not limited by the form of the computer-readable media on which the instructions of the inventive process are stored. For example, the instructions may be stored on CDs, DVDs, in FLASH memory, RAM, ROM, PROM, EPROM, EEPROM, hard disk or any other information processing device with which the computer system 600 communicates, such as a server or computer. The general purpose storage controller 612 connects the storage medium disk 614 with communication bus 610, which may be an ISA, EISA, VESA, PCI, or similar, for interconnecting all of the components of the computer system 600.

One or more processors in a multi-processing arrangement may also be employed to execute the sequences of instructions contained in main memory 604. In alternative embodiments, hard-wired circuitry may be used in place of or in combination with software instructions. Thus, embodiments are not limited to any specific combination of hardware circuitry and software.

As stated above, the computer system 600 includes at least one computer readable medium or memory for holding instructions programmed according to the teachings of the invention and for containing data structures, arrays, matrices, tables, records, or other data described herein.

Stored on any one or on a combination of computer readable media, the present invention includes software for controlling the computer system 600, for driving a device or devices for implementing the invention, and for enabling the computer system 600 to interact with a human user. Such software may include, but is not limited to, device drivers, operating systems, development tools, and applications software. Such computer readable media further includes the computer program product of the present invention for performing all or a portion (if processing is distributed) of the processing performed in implementing the invention.

The computer code devices of the present invention may be any interpretable or executable code mechanism, including but not limited to scripts, interpretable programs, dynamic link libraries (DLLs), Java classes, and complete executable programs. Moreover, parts of the processing of the present invention may be distributed for better performance, reliability, and/or cost.

The term “computer readable medium” as used herein refers to any medium that participates in providing instructions to the processor 602 for execution. A computer readable medium may take many forms, including but not limited to, non-volatile media, volatile media, and transmission media. Non-volatile media includes, for example, optical, magnetic disks, and magneto-optical disks, such as the hard disk 614 or a removable media drive. Volatile media includes dynamic memory, such as the main memory 604. Transmission media includes coaxial cables, copper wire and fiber optics, including the wires that make up the bus 610. Transmission media also may also take the form of acoustic or light waves, such as those generated during radio wave and infrared data communications.

Various forms of computer readable media may be involved in carrying out one or more sequences of one or more instructions to processor 602 for execution. For example, the instructions may initially be carried on a magnetic disk of a remote computer. The remote computer can load the instructions for implementing all or a portion of the present invention remotely into a dynamic memory and send the instructions over a telephone line using a modem. A modem local to the computer system 600 may receive the data on the telephone line and use an infrared transmitter to convert the data to an infrared signal. An infrared detector coupled to the bus 610 can receive the data carried in the infrared signal and place the data on the bus 610. The bus 610 carries the data to the main memory 604, from which the processor 602 retrieves and executes the instructions. The instructions received by the main memory 604 may optionally be stored on hard disk 614 either before or after execution by processor 602.

The computer system 600 of FIG. 6 also includes a network controller 608, such as an Intel Ethernet PRO network interface card from Intel Corporation of America, for interfacing with communication network 624. As can be appreciated, the communication network 624 can be a public network, such as the Internet, or a private network such as an LAN or WAN network, or any combination thereof and can also include PSTN or ISDN sub-networks. The communication network 624 can also be wired, such as an Ethernet network, or can be wireless such as a cellular network including EDGE, 3G and 4G wireless cellular systems. The wireless network can also be WiFi, Bluetooth, or any other wireless form of communication that is known.

An input/output (I/O) interface 616 interfaces with a display screen 618 (output peripheral device), a mouse 620 and/or a keyboard 622 (input peripheral devices). In certain embodiments when the display screen 618 is integrated with touch technology, the display screen 618 can also function as an input peripheral device. More peripheral devices, not shown in FIG. 6, may be connected to the I/O interface 616 to be added to the computer system 600, such as loudspeakers, a microphone and a printer.

Thus, the foregoing discussion discloses and describes merely exemplary embodiments of the present invention. As will be understood by those skilled in the art, the present invention may be embodied in other specific forms without departing from the spirit or essential characteristics thereof. Accordingly, the disclosure of the present invention is intended to be illustrative, but not limiting of the scope of the invention, as well as other claims. The disclosure, including any readily discernible variants of the teachings herein, defines, in part, the scope of the foregoing claim terminology such that no inventive subject matter is dedicated to the public.

1. A computer-implemented process of evaluating the psychological status of a subject human, comprising:
comparing the subject human's arrangements of a prescribed group of colors to one or more reference arrangements of the prescribed group of colors by one or more human references;
wherein the subject human's arrangements of the prescribed group of colors represents the subject human's perception of at least one pair of words with opposite meanings;
wherein the subject human's arrangements of the prescribed group of colors include first and second subject arrangements of colors;
wherein the reference arrangements of the prescribed group of colors include first and second reference arrangements of colors;
wherein the comparing comprises the following steps:
instructing the subject human to arrange the prescribed group of colors to reflect a positive emotional state or a favorable word to obtain the first subject arrangement of colors;
instructing the subject human to arrange the prescribed group of colors to reflect a negative emotional state that is opposite to the positive emotional state or a unfavorable word that is opposite to the favorable word to obtain the second subject arrangement of colors;
associating each color in the prescribed group of colors with a numerical code to obtain first and second subject arrangements of numerical codes from the first and the second subject arrangements of colors and first and second reference arrangements of numerical codes from the first and the second reference arrangements of colors; and
deriving a standard deviation and/or a linear correlation coefficient based on the differences between the first and the second subject arrangements of numerical codes and the first and the second reference arrangements of numerical codes, respectively.

2. The computer-implemented process of claim 1, wherein the one or more human references comprise a population of psychologically healthy humans.

3. The computer-implemented process of claim 1, wherein the first and the second subject arrangements of numerical codes and the first and the second reference arrangements of numerical codes are each arranged in an array.

4. The computer-implemented process of claim 3, wherein the differences are based on the differences in the locations of the numerical codes in the arrays.

5. The computer-implemented process of claim 1, wherein a standard deviation of greater than 2 is indicative of an abnormal psychological status.

6. The computer implemented process of claim 1, wherein a linear correlation coefficient of less than 0.95 is indicative of an abnormal psychological status.

7. A non-transitory computer-readable storage medium having computer-readable instructions thereon which when executed by a computer case the computer to perform a computer-implemented process of evaluating the psychological status of a subject human, comprising:
comparing the subject human's arrangements of a prescribed group of colors to one or more reference arrangements of the prescribed group of colors by one or more human references;
wherein the subject human's arrangements of the prescribed group of colors represents the subject human's perception of at least one pair of words with opposite meanings;
wherein the subject human's arrangements of the prescribed group of colors include first and second subject arrangements of colors;
wherein the reference arrangements of the prescribed group of colors include first and second reference arrangements of colors;
wherein the comparing comprises the following steps:
instructing the subject human to arrange the prescribed group of colors to reflect a positive emotional state or a favorable word to obtain the first subject arrangement of colors;
instructing the subject human to arrange the prescribed group of colors to reflect a negative emotional state that is opposite to the positive emotional state or a unfavorable word that is opposite to the favorable word to obtain the second subject arrangement of colors;
associating each color in the prescribed group of colors with a numerical code to obtain first and second subject arrangements of numerical codes from the first and the second subject arrangements of colors and first and second reference arrangements of numerical codes from the first and the second reference arrangements of colors; and
deriving a standard deviation and/or a linear correlation coefficient based on the differences between the first and the second subject arrangements of numerical codes and the first and the second reference arrangements of numerical codes, respectively.

8. The non-transitory computer-readable storage medium of claim 7, wherein the one or more human references comprise a population of psychologically healthy humans.

9. The non-transitory computer-readable storage medium of claim 7, wherein the first and the second subject arrangements of numerical codes and the first and the second reference arrangements of numerical codes are each arranged in an array.

10. The non-transitory computer-readable storage medium of claim 9, wherein the differences are based on the differences in the locations of the numerical codes in the arrays.

11. The non-transitory computer-readable storage medium of claim 7, wherein a standard deviation of greater than 2 is indicative of an abnormal psychological status.

12. The non-transitory computer-readable storage medium of claim 7, wherein a linear correlation coefficient of less than 0.95 is indicative of an abnormal psychological status.

13. A computer system having at least one processor module and at least one memory unit communicatively coupled to the processor module, the memory module configured to store one or more modules that, when executed by the processor module, are operative to perform a computer-implemented process of evaluating the psychological status of a subject human, comprising:
comparing the subject human's arrangements of a prescribed group of colors to one or more reference arrangements of the prescribed group of colors by one or more human references;
wherein the subject human's arrangements of the prescribed group of colors represents the subject human's perception of at least one pair of words with opposite meanings;
wherein the subject human's arrangements of the prescribed group of colors include first and second subject arrangements of colors;
wherein the reference arrangements of the prescribed group of colors include first and second reference arrangements of colors;
wherein the comparing comprises the following steps:
instructing the subject human to arrange the prescribed group of colors to reflect a positive emotional state or a favorable word to obtain the first subject arrangement of colors;
instructing the subject human to arrange the prescribed group of colors to reflect a negative emotional state that is opposite to the positive emotional state or a unfavorable word that is opposite to the favorable word to obtain the second subject arrangement of colors;
associating each color in the prescribed group of colors with a numerical code to obtain first and second subject arrangements of numerical codes from the first and the second subject arrangements of colors and first and second reference arrangements of numerical codes from the first and the second reference arrangements of colors; and
deriving a standard deviation and/or a linear correlation coefficient based on the differences between the first and the second subject arrangements of numerical codes and the first and the second reference arrangements of numerical codes, respectively.

14. The computer system of claim 13, wherein the one or more human references comprise a population of psychologically healthy humans.

15. The computer system of claim 13, wherein the first and the second subject arrangements of numerical codes and the first and the second reference arrangements of numerical codes are each arranged in an array.

16. The computer system of claim 15, wherein the differences are based on the differences in the locations of the numerical codes in the arrays.

17. The computer system of claim 13, wherein a standard deviation of greater than 2 is indicative of an abnormal psychological status.

18. The computer system of claim 13, wherein a linear correlation coefficient of less than 0.95 is indicative of an abnormal psychological status.

19. The computer system of claim 13, further comprising one or more peripheral devices.
